(19) United States
(12) Patent Application Publication
(10) Pub. No.: US 2007/0134169 A1
(43) Pub. Date: Jun. 14, 2007

(54) Methods for smoking cessation or alcohol cessation or other addiction cessation

(76) Inventor: Michael D Rabinoff, San Jose, CA (US)

Correspondence Address: JEFFREY FURR, 253 N. MAIN STREET, JOHNSTOWN, OH, 43031, US

(21) Appl. No.: 11/609,265

(22) Filed: Dec. 11, 2006

Related U.S. Application Data
(60) Provisional application No. 60/597,573, filed on Dec. 11, 2005.

(51) Int. Cl. A61K 48/00; A61K 9/68; A61M 11/00; A61K 9/127

(52) U.S. Cl. 424/48; 424/449; 424/450; 128/200.23; 514/44

(57) ABSTRACT

The invention is a patch or other applicator, or other method of administration, and a process to concept is to utilize patches (or other methods of delivery), that may contain nicotine in addition to, and that contain other chemicals that have been shown, or will be shown, either by subjective or objective measures, to increase “impact” (an tobacco industry term for the subjective awareness by the smoker of the drug effects of nicotine), or to increase addiction. Chemicals that decrease addiction behavior, or decrease signs and symptoms of withdrawal from the addictive substance or behavior may also be included. The claimed methods have application in the treatment of addiction to tobacco products, addiction to alcohol, and other addictions.

### BACKGROUND OF INVENTION

This invention relates to a method for smoking cessation or alcohol cessation or other addiction cessation.

### 1. Background

Diseases related to cigarette smoking, like lung disease, heart disease and cancer, claim an estimated 400,000 lives each year. The combustion of tobacco produces poisons and carcinogens that present a significant health hazard for smokers and non-smokers alike. Nicotine is a principal component of tobacco is very physically addictive, making it very difficult for a smoker to quit.

The process of smoking a cigarette, pipe or cigar delivers nicotine vapors to the lungs, where nicotine is absorbed through the arteries and delivered to the brain. Nicotine interacts with nicotine cholinergic receptors in the brain to induce the release of neurotransmitters and produce an immediate reward—the “rush” that smokers experience—that is associated with a rapid rise in blood level. A persistent stimulus is also produced, and is associated with a high blood level of nicotine. As such, the dopaminergic reward system is activated which eventually results in nicotine dependency.

The behavioral and social aspects of smoking, e.g., the hand-to-mouth ritual, etc., are also habit-forming.

There are other addictions as well to alcohol and drugs which have a detrimental affect on society.

There have been a large number of anti-smoking programs and devices. None of these have worked as effectively as desired with many going back to smoking. This is based on understanding that the tobacco industry added other addictive agents to cigarettes. This may explain why nicotine patches as well as other means have been found to be minimally effective for smoking prevention. (if there are multiple addictive chemicals in smoke, and you only replace one, then the craving for the other compounds isn't being treated).

There is still room for improvement in the art.

### SUMMARY OF INVENTION

The invention is a patch or other applicator, or other method of administration, and a process to concept is to utilize patches (or other methods of delivery), that may contain nicotine in addition to, and that contain other chemicals that have been shown, or will be shown, either by subjective or objective measures, to increase “impact” (an tobacco industry term for the subjective awareness by the smoker of the drug effects of nicotine), or to increase addiction. Chemicals that decrease addiction behavior, or decrease signs and symptoms of withdrawal from the addictive substance or behavior may also be included. The claimed methods have application in the treatment of addiction to tobacco products, addiction to alcohol, and other addictions.

### BRIEF DESCRIPTION OF DRAWINGS

Without restricting the full scope of this invention, the preferred form of this invention is illustrated in the following drawings:

FIG. 1 displays a transdermal patch delivery system;

FIG. 2 displays a gum delivery system;

FIG. 3 displays an inhaler delivery system; and

FIG. 4 displays a nasal spray.

### DETAILED DESCRIPTION

The following description is demonstrative in nature and is not intended to limit the scope of the invention or its application of uses.

The current invention is a novel medication treatment for smoking cessation or alcohol cessation or other addiction. It is a new combination patch formulations for smoking cessation or alcohol cessation. This is based on understanding that the tobacco industry added other addictive agents to cigarettes. This may explain why nicotine patches have been found to be minimally effective for smoking prevention. (if there are multiple addictive chemicals in smoke, and you only replace one, then the craving for the other compounds isn't being treated).

The current invention is a patch and a process to concept is to utilize patches (or other methods of delivery), that contain not only nicotine, but other chemicals that have been shown, or will be shown, either by subjective or objective measures, to increase “impact” (an tobacco industry term for the subjective awareness by the smoker of the drug effects of nicotine), or to increase addiction.

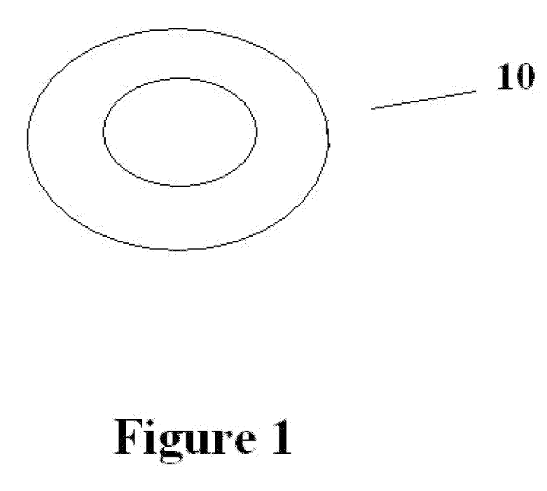

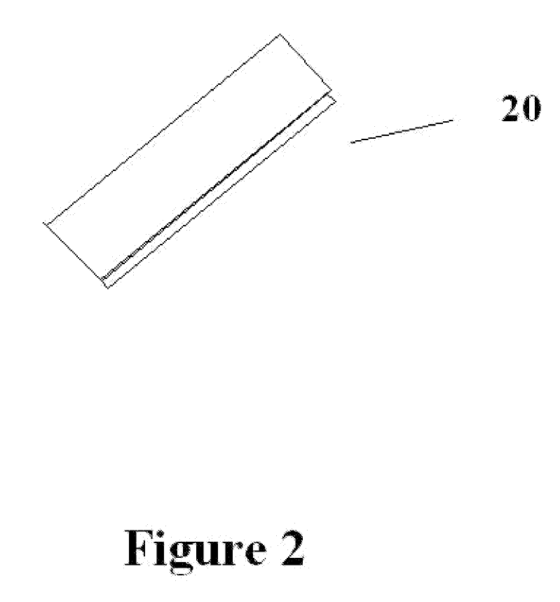

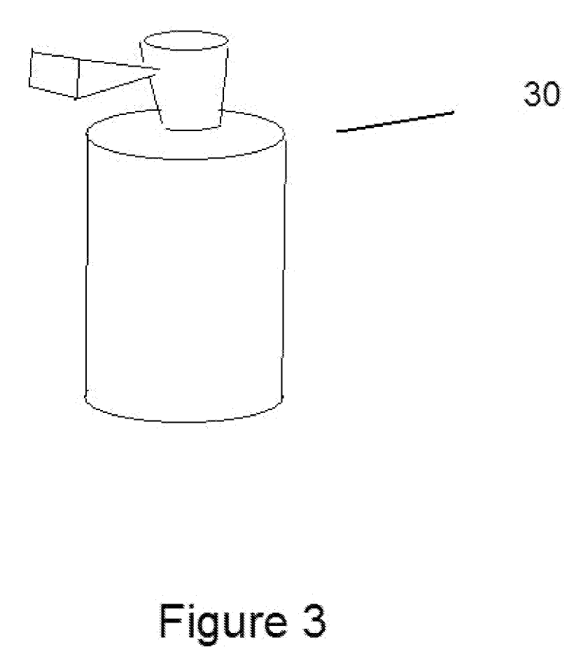

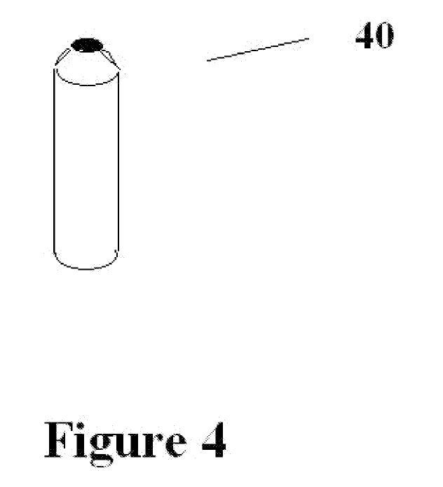

Addition of acetaldehydes and/or isoquinolones or isoquinoxalines or tetrahydro isoquinolones (5,6,7,8-tetrahydroquin-oxaline which has been documented to be added to cigarettes by a tobacco company, or salsinol or tetrahydropapaveroline, etc.) and/or pyridine or pyridine derivatives (addictive compounds in cigarette smoke) and/or lobeline (agent to act as potential replacement for nicotine) to nicotine transdermal patches 10 (or gum 20 or inhaler 30 or nasal spray 40 or other applicator) to improve cessation results. These applicators are shown in FIGS. 1, 2, 3, and 4. Tetrahydroquinolines (TIQs), based on experimental data, have been hypothesized to act as “false neurotransmitters” in catecholamine containing neurons. In the 1960s, formaldehyde was shown to condense with endogenous catecholamines to form TIQs. That acetaldehyde is highly reactive with catecholamines was one of the reasons for DeNoble pursuing his research on the reinforcing effects of acetaldehyde, and he showed that rats would bar press for acetaldehyde or nicotine, and have a synergistic addictive effect with increased bar pressing with the administration of both compounds. It is thought that TIQs can serve as a “false neurotransmitter”, and have an addictive effect.

This can be used in treating alcohol abuse or dependence (such as in a patch with acetaldehyde, or acetaldehydes, and/or naltrexone and/or other opioid antagonists and/or isoquinolones and/or isoquinoxalines). When alcohol is metabolized it forms acetaldehyde, and it has been hypothesized that the acetaldehyde interacts with catecholamines in the brain to from TIQs which then stimulate opioid pathways leading to euphoric feelings.

Regarding other addictions, a product that provides a euphoric feeling while the user is undergoing withdrawal, might help in the cessation process.

Other agents possibly enhancing addiction in cigarettes, and which can be used in smoking cessation patches, include levulinic acid (4-oxopentanoic acid)+nicotine and nicotine levulinate to enhance binding of nicotinic receptors (low nanomolar ranges); and also the possible use of 4-pentenoic acid, omega-pentadecalactone, 2,3-pentanedione, 2-pentanone, 2-pentylpyridine (other known cigarette additives).

Other chemicals that can possibly enhance addiction, and which can be used in smoking cessation patches, are chemicals that were shown by cigarette companies or other research, either by subjective testing measures, or objective measures (such as CNS P1-N2 amplitudes, but including other measures, but not limited to, behavioral, neurological, biochemical, gene or protein sequence or expression product scientific measures/markers) to increase “impact” or addiction: for instance, tobacco industry research showed P1-N2 amplitudes, an objective measure of “impact”, were increased by pyrazine (already mentioned), vanillin and propylene glycol. Thus, these chemicals could also be added to a patch (or other method of delivery) to decrease “impact”, and which might help the smoker, as a replacement therapy, to cease smoking, or to switch to/substitute with use of a reduced harm or non-harmful replacement for cigarettes.

Smoking is known to inhibit monoamineoxidase A and B. Chemicals that inhibit monoamineoxidase A and B, or either alone, may be useful in facilitating smoking cessation. This could occur by allowing some of the physiological effects of cigarette smoking to occur, while a device and/or methods treat the smoking addiction with nicotine and/or other addictive compound, or compounds, replacement.

Smoking cessation or alcohol cessation or other addiction cessation are known to have a significant withdrawal syndrome for many smokers or alcohol abusing or dependent individuals or other substance or substances addicted individuals. Treatment to ameliorate or eliminate withdrawal signs and symptoms may also improve smoking cessation or alcohol cessation or other addiction cessation.

Dosing of chemicals in patch (first model with nicotine plus acetaldehyde), plus regular patch technology, or liposome patch technology, or Noven patch technology, or other patch technology, including methods to enhance chemicals crossing the blood brain barrier, including liposomal, CNS and specific CNS region targeted liposomal (via compounds on outer perimeter of liposome) or other methods of targeting the CNS, including, but not limited to bound chemicals that easily cross the blood-brain barrier, and micro sized formulations).

All publications and patent applications cited in this specification are herein incorporated by reference as if each individual publication or patent application were specifically and individually indicated to be incorporated by reference.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A device for treating addiction comprising:
an applicator that delivers a plurality of chemicals

2. A device according to claim 1, wherein said delivery is by one or more of a set of, oral ingestion, sublingual, via saliva, intra-muscular administration, intra-venous administration, intra-arterial administration, intra-thecal administration, trans-vaginal, trans-rectal, trans-urethral or trans-vesicular, or trans-cutaneous.

3. A device according to claim 2, in which said delivery means include one or more of a set of; liposomal, microsomal, DNA-conjugate, RNA-conjugate, siRNA-conjugate, chemical-conjugate, viral or viral fragment CNS targeting agent; specific CNS region targeted liposomal, microsomal, DNA-conjugate, RNA-conjugate, siRNA-conjugate, chemical-conjugate, viral or viral fragment CNS targeting agent; or micro sized formulations.

4. A device according to claim 1, wherein said chemical include one or more of a set of acetaldehyde, acetaldehyde related compounds, formaldehyde related compounds and derivatives, isoquinolones, isoquinoxalines, tetrahydro-isoquinolones (and related compounds), 1,2,3,4-tetrahydroisoquinoline (and related compounds), 5-tetrahydroquin-oxaline, 6-tetrahydroquin-oxaline, 7-tetrahydroquin-oxaline, 8-tetrahydroquin-oxaline, 5,6,7,8-tetrahydroquin-oxaline, salsinol, tetrahydropapaveroline, pyridine, pyridine derivatives, lobeline, levulinic acid, 4-oxopentanoic acid, nicotine levulinate, 4-pentenoic acid, omega-pentadecalactone, 2,3-pentanedione, 2-pentanone, 2-pentylpyridine, pyrazine (and related compounds), vanillin, propylene glycol, naltrexone, opioid agonists, opioid antagonists, opioid partial agonists, antagonists, harman, norharman, carbolines (and related compounds), beta-carbolines (and related compounds), aromatic amines, nicotine, or nicotine related compounds.

5. A device according to claim 1, which includes a means to enhance chemicals crossing the blood brain barrier.

6. A device according to claim 5, in which said delivery means include one or more of a set of; liposomal, microsomal, DNA-conjugate, RNA-conjugate, siRNA-conjugate, chemical-conjugate, viral or viral fragment CNS targeting agent; specific CNS region targeted liposomal, microsomal, DNA-conjugate, RNA-conjugate, siRNA-conjugate, chemical-conjugate, viral or viral fragment CNS targeting agent; or micro sized formulations.

7. A device according to claim 2, wherein said device is a transdermal patch.

8. A device according to claim 2, wherein said device is gum.

9. A device according to claim 2, wherein said device is an inhaler.

10. A device according to claim 2, wherein said device is a nasal spray.

11. A method of administration for treating addiction comprising:
a method of administration dosage to the body where delivery is by one or more of a set of, oral ingestion, sublingual, via saliva, intramuscular administration, intra-venous administration, intra-arterial administration, intra-thecal administration, trans-vaginal, trans-rectal, trans-urethral or trans-vesicular, or trans-cutaneous.

12. A method according to claim 11, in which said delivery means include one or more of a set of; liposomal, microsomal, DNA-conjugate, RNA-conjugate, siRNA-conjugate, chemical-conjugate, viral or viral fragment CNS targeting agent; specific CNS region targeted liposomal, microsomal, DNA-conjugate, RNA-conjugate, siRNA-conjugate, chemical-conjugate, viral or viral fragment CNS targeting agent; or micro sized formulations.

13. A method according to claim 11, wherein said chemical include one or more of a set of acetaldehyde, acetaldehyde related compounds, formaldehyde related compounds and derivatives, isoquinolones, isoquinoxalines, tetrahydro-isoquinolones, (and related compounds), 1,2,3,4-tetrahydroisoquinoline (and related compounds), 5-tetrahydroquin-oxaline, 6-tetrahydroquin-oxaline, 7-tetrahydroquin-oxaline, 8-tetrahydroquin-oxaline, 5,6,7,8-tetrahydroquin-oxaline, salsinol, tetrahydropapaveroline, pyridine, pyridine derivatives, lobeline, levulinic acid, 4-oxopentanoic acid, nicotine levulinate, 4-pentenoic acid, omega-pentadecalactone, 2,3-pentanedione, 2-pentanone, 2-pentylpyridine, pyrazine (and related compounds), vanillin, propylene glycol, naltrexone, opioid agonists, opioid antagonists, opioid partial agonists, antagonists, harman, norharman, carbolines (and related compounds), beta-carbolines (and related compounds), aromatic amines, nicotine, or nicotine related compounds.

14. A method according to claim 11, which includes a means to enhance chemicals crossing the blood brain barrier.

15. A method according to claim 11, wherein a transdermal patch is used as a delivery method.

16. A method according to claim 11, wherein gum is used as a delivery method.

17. A method according to claim 11, wherein an inhaler is used as a delivery method.

18. A method according to claim 11, wherein a nasal spray is used as a delivery method.

19. A device according to claim 1, wherein said chemical include one or more of a set of chemical or chemicals known to decrease addiction behavior, or smoking behavior, or facilitate smoking cessation, or facilitate addiction cessation, or decrease signs and symptoms of nicotine and cigarette withdrawal, or other withdrawal, may be included in the device, including (but not limited to), clonidine, amfebutamone, mecamylamine, nortriptyline and moclobemide, dextrose, sugars, flavorants, vitamins, amino acids, minerals, other neutraceuticals, selegiline, pargyline, clorgyline, tranylcypromine, phenelzine, other inhibitors of MAO-A, other inhibitors of MAO-B, p-tyramine, serotonin, beta-phenylethylamine, or compounds related to these compounds.

20. A device according to claim 11, wherein said chemical include one or more of a set of chemical or chemicals known to decrease addiction behavior, or smoking behavior, or facilitate smoking cessation, or facilitate addiction cessation, or decrease signs and symptoms of nicotine and cigarette withdrawal, or other withdrawal, may be included in the device, including (but not limited to), clonidine, amfebutamone, mecamylamine, nortriptyline and moclobemide, dextrose, sugars, flavorants, vitamins, amino acids, minerals, other neutraceuticals, selegiline, pargyline, clorgyline, tranylcypromine, phenelzine, other inhibitors of MAO-A, other inhibitors of MAO-B, p-tyramine, serotonin, beta-phenylethylamine, or compounds related to these compounds.
